(19) United States
(12) Patent Application Publication
(10) Pub. No.: US 2003/0007959 A1
(43) Pub. Date: Jan. 9, 2003

(54) Oral methods of treatment

(76) Inventors: John Lezdey, Indian Rocks Beach, FL (US); Darren Lezdey, Indian Rocks Beach, FL (US)

Correspondence Address: JOHN LEZDEY, JOHN LEZDEY AND ASSOCIATES, 4625 EAST BAY DRIVE, SUITE 302, CLEARWATER, FL, 33764, US

(21) Appl. No.: 09/885,654

(22) Filed: Jun. 20, 2001

(51) Int. Cl. A61K 38/43; A61K 39/395; A61K 31/57; A61K 38/00

(52) U.S. Cl. 424/94.1; 424/131.1; 514/12; 514/171

(57) ABSTRACT

A method is provided for the treatment of pulmonary diseases and acquired immune disease by the oral administration of a stabilized protease inhibitor that transgresses the gastrointestinal tract. Crystalline crosslinked and polyethylene glycol adducts can be used as well as coated protease inhibitors and coated stabilized protease inhibitors.

### FIELD OF THE INVENTION

The present invention relates to the treatment of pulmonary diseases by the oral administration of proteins that possess an inhibitory activity against neutrophil elastase or can maintain a proteinase-antiproteinase balance. More particularly, it is provided an oral medicament that transgresses the gastrointestinal pathway to deliver an effective amount of medicament to the lower respiratory tract. The compositions can also be used in the treatment of AIDS.

### BACKGROUND OF THE INVENTION

The treatment of emphysema, both hereditary and acquired, with alpha 1-antitrypsin (AAT) and secretory leucocyte protease inhibitor (SLPI) by infuision and by inhalation therapy are well known. U.S. Pat. No. 5,093,316 to Lezdey et al, which is herein incorporated by reference, discloses the administration by aerosolization of alpha 1-antitrypsin and that it can be administered orally. However, there is not disclosed any means for delivering the drug without its destruction in the gastrointestinal tract.

Cystic fibrosis is a neutrophil elastase related disease that can also be treated by aerosolizing alpha 1-antitrypsin. The oral administration of AAT for cystic fibrosis was found to have also failed.

The infusion of AAT results in the drug going to the lower respiratory tract where destruction of the lung by neutrophil elastase takes place in both smokers and non-smokers with emphysema. Inhalation therapy primarily works in the upper respiratory tract.

Treatment of emphysema by infusion is costly, painful and time consuming. A method of administration of an anti-elastase drug so that it goes into the blood stream and is administered to the lower respiratory tract without infusion would be useful.

In many pulmonary diseases including asthma and emphysema, corticosteroids are commonly utilized either by oral or aerosol administration.

Additionally, it would be useful to provide a prophilaxsis to young smokers to maintain a proper proteinase-antiproteinase balance that would avoid their acquiring pulmonary disease, such as, emphysema, bronchitis and COPD.

For young children who have asthma, an oral medicament could be useful for administration while away from any medical facility.

Since oxidants are spontaneously released by alveolar macrophages of cigarette smokers, the uses of an anti-oxidant in combination with the medicament would ensure that a greater amount of medicament is present to be an effective inhibitor of neutrophil elastase.

There have been many methods proposed to stabilize proteins so that they can be administered orally. Crosslinking the protein, forming conjugates, and crystallization have all been suggested.

U.S. Pat. No. 5,880,255 to Delgade et al, which is herein incorporated by reference, discloses the preparation of polyethylene glycol (PEG)—protein which process can be used to prepare the adducts that transgress the gastrointestinal tract in the invention.

U.S. Pat. No. 6,004,549 to Reichert et al, which is herein incorporated by reference, disclose the preparation of crystalline protein and polyethylene glycol or vegetable oil, which can be used in the practice of the invention.

U.S. Pat. No. 5,554,730, which is herein incorporated by reference, discloses a process for preparing polysaccharide-protein conjugates.

Each of alpha 1-antitrypsin, secretory leucocyte protease inhibitor and alpha 2-macroglobulin has been disclosed by Lezdey et al as having anti-viral characteristics.

U.S. Pat. No. 6,140,475 to Margolin, which is herein incorporated by reference, discloses the preparation of crosslinked crystalline proteins. The process disclosed can be used for the preparation of the compositions used in the present invention.

Protein crystals can be grown by the controlled crystallization of protein out of aqueous solution or aqueous solution-containing organic solvents. Conditions to be controlled include, for example, the rate of evaporation of solvent, the presence of appropriate co-solutes and buffers, pH and temperature. A comprehensive review of the various factors affecting the crystallization of proteins has been published by McPherson, Methods Enzymol., 114, pp. 112-20 (1985).

McPherson and Gilliland, J. Crystal Growth, 90, pp. 51-59 (1988) have compiled comprehensive lists of proteins and nucleic acids that have been crystallized, as well as the conditions under which they were crystallized. A compendium of crystals and crystallization recipes, as well as a repository of coordinates of solved protein and nucleic acid structures, is maintained by the Protein Data Bank at the Brookhaven National Laboratory [http//www.pdb.bnl.gov; Bernstein et al., J. Mol. Biol., 112, pp. 535-42 (1997)]. These references can be used to determine the conditions necessary for crystallization of a protein, as a prelude to the formation of an appropriate crosslinked protein crystal, and can guide the crystallization strategy for other proteins. Alternatively, an intelligent trial and error search strategy can, in most instances, produce suitable crystallization conditions for many proteins, provided that an acceptable level of purity can be achieved for them [see, e.g., C. W. Carter, Jr. and C. W. Carter, J. Biol. Chem., 254, pp. 12219-23 (1979)].

### SUMMARY OF THE INVENTION

The invention provides for the oral delivery of a protein that can be used in the treatment of a pulmonary disease and/or an acquired immune deficiency disease (AIDS). More particularly, there is provided a protein that not only helps to maintain a proteinase-antiproteinase balance that affects the respiratory tract but prevents the proliferation of HIV in a mammal.

Accordingly, there is provided the crosslinked, crystalline, conjugate or encapsulated proteins selected from the group consisting of alpha 1-antitrypsin, alpha 2-macroglobulin and secretory leucocyte protease inhibitor in a form so as to transgress the gastrointestinal track.

The compositions of the invention can also include anti-oxidants such as glutathione, catalase, superoxide dismutase, mannitol, methionine, and the like, especially in connection with smokers to prevent inactivation by oxidants such as myeloperoxidase.

Among the inflammatory conditions which may be treated with the protease inhibitors, there are included inflammations of the tracheobronchial tree such as asthma, emphysema, chronic obstructive pulmonary disease and chronic granulomatous lung disease i.e. Sarcoid.

The use of alpha 1-antitrypsin is especially useful in the treatment of the various inflammatory lung conditions including those which are induced by smoking or genetically deficient alpha 1-antitrypsin diseases.

The combination of other serine protease inhibitors such as secretory leucocyte protease inhibitor and/or alpha 2-macroglobulin provides a broader spectrum for treating associated symptoms of inflammation. The combination is especially useful in the treatment of AIDS.

The genetic mutagenesis of the serine protease inhibitors may be performed utilizing conventional techniques associated with other genes without affecting the utility of the compound. In AIDS, it is believed that the disease injures the liver and reduces the body's production of AAT so as to result in the skin lesions and pulmonary complications.

It is the object of the invention to provide an anti-inflammatory composition which can be used as a prophylaxis and/or in treatment of existing inflammatory pulmonary diseases, particularly, emphysema and cystic fibrosis.

It is another object of the invention to provide a composition for smokers which can prevent or treat irritations arising from tobacco use.

It is a further object of the invention to provide an anti-inflammatory composition for pulmonary diseases, which is well tolerated by the human body and is free of side effects.

It is yet a further object of the invention to provide a composition for use in the treatment of AIDS and its symptoms.

### DETAILED DESCRIPTION OF THE PRESENT INVENTION

The object of the present invention can be achieved by the oral administration of protease inhibitors in suitable pharmaceutical form to patients suffering from inflammatory conditions of the pulmonary tract. The composition can also be used for the treatment of AIDS.

The present invention provides a pharmaceutical composition which comprises the protease inhibitor alone or in combination with anti-oxidants.

Normally between 50 and 100 mg of the compositions of the invention will be administered each day of treatment (to an average 70 kg adult). Similar amounts may be administered to prevent the occurrence of the condition.

In the treatment of chronic cases of inflammatory lung conditions wherein the cells express proteases, such as in the case of cystic fibrosis and emphysema, the patient is typically administered intravenously 6 to 90 mg of the protease inhibitor compound per kilogram of body weight daily. The treatment is continued for a period of time until there is a reversal of the biochemical abnormalities in serum and lung fluid that characterizes the disorder. The treatment may be followed up by administration of the drug by inhalation to more rapidly promote healing. For use in the prevention of the disease, the drug should be administered orally on a daily basis. This daily treatment is believed to aid smokers from incurring the inflammation of the pulmonary tract which is commonly associated with smoking.

Patients with emphysema and asthma usually include a corticosteroid in their regimen. The corticosteroid can be taken together or separately with the protease inhibitor.

According to one embodiment of the present invention, an orally deliverable formulation enabling the therapeutic delivery of a protease inhibitor of interest, the site of action for the protease inhibitor of interest being portions of the gastrointestinal tract encountered following transit of the formulation through the stomach. The initial step of the formulation method requires the production of a multiparticulate dosage core particle. The multiparticulate dosage core particle is made up of 3 components, the total weight of the 3 components in dry form defining a batch size.

One of the three components referred to above is polyethylene glycol (PEG). An aqueous PEG solution is prepared. In preferred embodiments, the dry weight of the PEG component represents from about 2.5% to about 15% of the batch size (weight/weight), and the water component of the aqueous PEG solution represents approximately 30-60% of the batch size (weight/weight). Preferably, PEG 4,000-PEG 8,000 is employed in connection with the present formulation.

Cigarette smoke accounts for producing over 90% of the inflammatory cells in a smokers lungs, which are a result of increases in macrophages. The macrophages cause the release of a metalloproteinase that solubilizes many extracellular matrix proteins, including elastase. These metailoproteinases generate monocyte chemotactic activity. Thus, macrophage-mediated lung destruction after exposure to cigarette smoke is directly related to the metalloproteinase, which can result in bronchitis, COPD and asthma. Inactivation of the metalloproteinases at an early stage is advisable to prevent the large proteinase-anti-proteinase imbalance that leads to lung destruction.

A homogenous mixture of the protease inhibitor and rnicrocrystalline cellulose, both in dry form, is then prepared. In preferred embodiments, the protease inhibitor represents from about 50% to about 95% of the batch size (weight/weight). Microcrystalline cellulose comprises from about 2.5% to about 35% of the batch size (weight/weight).

The adducts of the invention metabolize to the natural or recombinant protease inhibitors. Therefore, any form of oral administration of the protease inhibitors, which transgresses the gastrointestinal tract can be utilized. It is understood that there are many methods of stabilizing the protease inhibitors to pass through the gastrointestinal tract. The stabilization is also useful to provide a longer shelf life to the protease inhibitors.

It is further understood that the amount of protease inhibitor to be administered is dependant upon the age, weight and severity of condition. The compositions are especially useful in the early stages of the disease so as to maintain an early protease-antiprotease balance.

The following examples are only representations of preferred methods of producing the stabilized protease inhibitors.

### EXAMPLE 1

A tablet having a clear coating is prepared as follows.

A. Tablets are prepared on a TF-MINI roller compactor apparatus sold by Vector Corp., which comprise 20 mg. secretory leucocyte protease inhibitor, in each tablet. The tablets are formed with the following inactive ingredients: mannitol, cellulose, hydroxypropyl methyl cellulose, polyethylene glycol, sodium starch glycolate and starch.

B. The tablets are coated in a Vector Hi-Coater perforated pan coating system with the following formulation:

| INGREDIENTS | WT |
|---|---|
| Eudragit ® | 100.0 g |
| Glidant (talc) | 7.5 g |
| Plasticizer | 3.0 g |
| Water | 92.0 g |

Eudragit® comprises 30 g of polymer and the remainder water.

The glidant, plasticizer and water are mixed in a separate vessel using a high shear mixer. This is then added to the Eudragit® dispersion and is mixed at low shear.

The glidant may vary from 25 to 100% by weight based on the solids of Eudragit®. The plasticizer may vary from 10 to 12% by weight based on the amount of polymer solids.

Glidants which may be used include silica, glycerol monostearate and natural kaolin.

Plasticizers which may be used include citrate esters, PEG, tuactin and DBS. The plasticizers are usually utilized in the range of about 10 to 12% by weight based on the solid polymers. The mannitol also serves as the antioxidant.

The coated tablet can be used to transgress the gastrointestinal tract for use in the treatment of pulmonary diseases such as cystic fibrosis and asthma.

The composition can be used to prevent or treat the onset of bronchitis and COPD in smokers.

In lieu of SLPI there may be used AAT or alpha 2-macroglobulin.

This type of composition is particularly suited for including a corticosteroid such as beta-methasone to prevent interaction between the drugs since the corticosteroid can be first coated and formed into pellets for use in combination in the treatment of asthma or emphysema.

### EXAMPLE 2

Crystalization of alpha 1-antitrypsin.

Following the procedure of U.S Pat. No. 6,140,475, 1 gram of alpha 1-antitrypsin (PROLASTIN) is dissolved in 100 ml of saline solution. 1 gram of celite is dissolved in 100 ml of distilled deionized water and filtered. Solid calcium acetate is added to a concentration of 5 mM Ca(CH₃COO)₂. The pH is adjusted to pH 5.5 with concentrated acetic acid. 200 ml of 30% solution of PEG-8000 is added to the mixture and cooled overnight and the resulting crystals filtered.

The crystals can be crosslinked using glutanaldehyde or pursuant to the procedure in Augervante Chemie Inl Ed. Eugl 35 p. 2056 (1996).

The resulting crystals can be formulated into a tablet and coated so as to transgress the gastrointestinal tract in the treatment of pulmonary diseases such as cystic fibrosis or asthma.

Alternatively, a crosslinked product can be used with a coating.

What is claimed is:

1. A method for the treatment of mammals suffering from a pulmonary disease or an autoimmune disease which comprises orally administering an effective amount of a composition of a protein, which has been stabilized so as to transgress the gastrointestinal tract, said protein being the conjugate, crosslinked or crystalline adduct of a member selected from the group consisting of alpha 1-antitrypsin, secretory leuocyte protease inhibitor and alpha 2-macroglobulin.

2. The method of claim 1 including an antioxidant.

3. The method of claim 2 wherein said antioxidant is selected from the group consisting of glutathione, catalase and mannitol.

4. The method of claim 1 wherein said protein is a polyethylene glycol-alpha 1-antitrypsin adduct.

5. The method of claim 1 wherein said protein is a polyethylene glycol-secretory leucocyte protease inhibitor adduct.

6. The method of claim 1 wherein said protease inhibitors are crystalline.

7. The method of claim 1 wherein said protease inhibitors are crosslinked.

8. The method of claim 1 wherein said protease inhibitors are conjugated.

9. The method of claim 8 wherein said protease inhibitor is alpha 1-antitrypsin conjugated with dextran.

10. The method of claim 1 wherein said composition includes a corticosteroid.

11. The method of claim 10 wherein said corticosteroid is a beta-methasone.

12. The method of claim 1 including the step of separately administering orally or by inhalation a corticosteroid.

13. The method of claim 1 wherein said pulmonary disease is cystic fibrosis.

14. The method of claim 1 wherein said disease is smoking acquired emphysema.

15. The method of claim 1 wherein said disease is bronchitis.

16. The method for the treatment of mammals suffering from an acquired immune disease which comprises orally administering an effective amount of a composition of a protein, which has been stabilized so as to transgress the gastrointestinal tract, said protein being the conjugate, crosslinked or crystalline adduct of a member selected from the group consisting of alpha 1-antitrypsin, secretory luecocyte protease inhibitor and alpha 2-macroglobulin.

17. The method of claim 16 wherein said protein is a polyethylene glycol-alpha 1-antitrypsin adduct.

18. The method of claim 16 wherein said protein is a polyethylene glycol-secretory leucocyte protease inhibitor adduct.

19. The method of claim 16 wherein said protease inhibitors are crystalline.

20. The method of claim 16 wherein said protease inhibitors are crosslinked.

21. The method of claim 16 wherein said protease inhibitors are conjugated.
